(19) United States
(12) Patent Application Publication
(10) Pub. No.: US 2011/0265797 A1
(43) Pub. Date: Nov. 3, 2011

(54) EXTENDABLE TRACHEAL TUBE

(75) Inventor: Paul Waldron, Galway (IE)

(73) Assignee: Nellcor Puritan Bennett LLC, Boulder, CO (US)

(21) Appl. No.: 12/771,166

(22) Filed: Apr. 30, 2010

(51) Int. Cl. A61M 16/04; C08G 63/183; C08F 110/02; C08G 71/04; C08F 136/06; C08F 14/14; C08F 136/08; C08F 14/06; C08F 110/06

(52) U.S. Cl. 128/207.14; 526/344; 528/308.1; 526/352.2; 526/351; 526/335; 526/291; 526/340.2; 528/44

(57) ABSTRACT

The present disclosure describes systems and methods that utilize an extendable tracheal tube system. The extendable tracheal tube system includes an extendable tracheal tube having a distal end portion and a proximal end portion. The proximal end portion and the distal end portion of the extendable tracheal tube are capable of moving axially relative to each other. An obturator is also provided that is capable of aiding in the intubation and/or extubation of the extendable tracheal tube. The use of the extendable tracheal tube system and methods enables the secure attachment of the extendable tracheal tube to a patient airway while allowing for certain movements of the patient, such as neck and head movements.

### BACKGROUND

The present disclosure relates generally to tracheal tubes and, more particularly, to novel extendable tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Tracheal tubes may be utilized to define a clear passageway for air, other gases, and medicaments to the trachea and lungs, thus providing an artificial airway for spontaneous or mechanical ventilation of a patient. Such tracheal tubes may include endotracheal tubes and tracheostomy tubes. Endotracheal tubes, for example, are typically introduced into the trachea and may be used to establish an open airway into the lungs. The proximal end of the endotracheal tube may include a connector to attach various devices such as ventilators, manual respirators, suctioning equipment, nebulizers, vaporizers, and so forth. The distal end of the tracheal tube may include an inflatable cuff suitable for creating a seal between the outside of the tube and the interior of the passage in which the tube or device is inserted. In this way, substances can only flow through the passage via the tube or other medical device, allowing a medical practitioner to maintain control over the type and amount of substances flowing into and out of the patient.

In certain circumstances, the endotracheal tube may be securely attached to the patient's mouth, for example, by using a tracheal tube holder and/or tape. However, if the patient's neck or jaw were to move or if the clinician where move the patient to another location, the movement may dislodge the cuff out of its original position. Such movement may require repositioning of the tube and cuff, and possibly removal and replacement of the tube.

Moreover, at times it may be useful to extend such tubes beyond the point at which they originally terminate outside the patient's mouth (or neck). Conventional tubes do not permit such adjustment without movement and/or replacement of the tube.

### BRIEF DESCRIPTION OF THE DRAWINGS

Advantages of the disclosed techniques may become apparent upon reading the following detailed description and upon reference to the drawings in which:

FIG. 1 illustrates embodiments of an extendable tracheal tube and an obturator;

FIG. 2 is a perspective view of an embodiment of extendable tracheal tube;

FIG. 3 is a schematic view of embodiments of a distal end portion and a proximal end portion of an extendable tracheal tube;

FIG. 4 is another schematic view of embodiments of a distal end portion and a proximal end portion of an extendable tracheal tube;

FIG. 5 is a schematic axial view of embodiments of a distal end portion and a proximal end portion of an extendable tracheal tube;

FIG. 6 is a perspective view of embodiments of an extendable tracheal tube and an obturator;

FIG. 7 is a schematic view of embodiments of a distal end portion and a proximal end portion of an extendable tracheal tube;

FIG. 8 is another schematic view of embodiments of a distal end portion and a proximal end portion of an extendable tracheal tube; and

FIG. 9 is yet another schematic view of embodiments of a distal end portion and a proximal end portion of an extendable tracheal tube.

FIG. 10 is still another schematic view of embodiments of a distal end portion and a proximal end portion.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube, such as an endotracheal tube, may be used to seal a patient's airway and provide positive pressure to the lungs when properly inserted into a patient's trachea. Positioning the tracheal tube at a desired position within the trachea, for example during endotracheal intubation, may improve the performance of the tracheal tube and reduce clinical complications. In particular, the distal inserted end of the endotracheal tube may be positioned in the patient's trachea at a location substantially between the patient's vocal cords and carina. If the tube cuff is not inserted far enough past the vocal cords, for example, the tube may become more easily dislodged. If the tube is inserted too far into the trachea, such as past the carina, then the tube may only function to adequately ventilate one of the lungs, rather than both. Thus, a well positioned and proper seal against the tracheal passageway allows a ventilator to perform efficiently.

Provided herein are extendable tracheal tube systems and methods that facilitate the placement of the tracheal tube relative to certain anatomical structures, and the maintenance of a proper seal once the tracheal tube has been so placed. Such extendable tracheal tubes may include embodiments capable of allowing for an extension and retraction of the tracheal tube in an axial direction while maintaining a secure placement at both the distal and proximal ends. That is the tracheal tube may be placed in the trachea and include embodiments that allow the tracheal tube to extend and retract axially in accordance with certain patient movements, such as movements of the head or neck, or movements resulting from relocating the patient. By maintaining a proper airway seal, the disclosed embodiments allow for increased efficiency and convenience in patient ventilation and airway management.

Further, the disclosed techniques allow for the customization of the tracheal tube length. Generally, tracheal tubes are available in a subset of sizes from which doctors may select the closest approximate size for a particular patient. The difference in tube sizes may generally reflect both differences in the length of the tube as well as different tube diameters. In particular, doctors may wish to select, for example, an endotracheal tube with an appropriate length in order to allow the tube to be easily inserted into the patient while providing a length suitable for connection to an external device, such as a ventilator. The disclosed embodiments allow for a tracheal tube capable of having an extendable or retractable length. Indeed, the clinician is able resin the tracheal tube length even after the patient has been intubated. Accordingly, the tracheal tube may be custom fit to comformably support any number of patient anatomies and patient types.

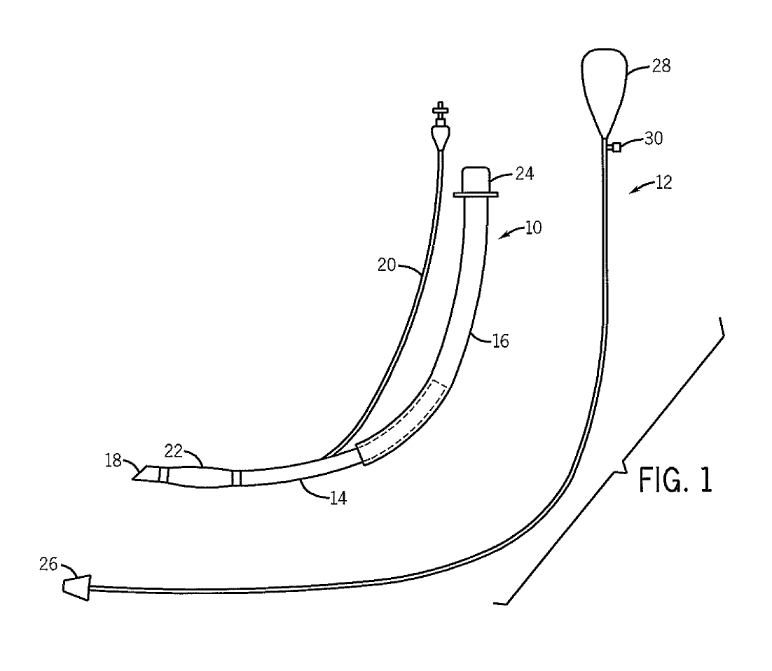

With the foregoing in mind and turning now to FIG. 1, the figure depicts an embodiment of a tracheal tube 10 and an obturator 12. The tracheal tube 10, shown here as an endotracheal tube, includes a distal end portion 14 for intubation into a patient, and a proximal end portion 16 for connection to other medical systems, such as a ventilator. The tracheal tube 10 is inserted through the mouth and into the trachea, and is typically curved so as to comformably fit inside the patient airway. A distal opening 18 may be beveled to allow for smoother insertion through the larynx and trachea. Accordingly, clinician may position the tracheal tube 10 at a desirable position in the patient's airway, for example, so that the distal opening 18 is substantially between the patient's vocal cords and the carina and not in the esophagus or mainstem bronchi.

The tracheal tube 12 may include any suitable number of lumens, such as lumen 20, that may be appropriately sized and shaped for inflation and deflation of an inflatable cuff 22, for suction and evacuation of fluids, and so forth. Once the tracheal tube 10 is placed at the desired location, the clinician may then use the lumen 20 to inflate the cuff 22. When inflated, the cuff 22 generally expands into the surrounding trachea to seal the tracheal passage around the tracheal tube 10, for example, to facilitate the controlled delivery of gases, medicines, and other substances, via a medical device (e.g., through the tube). The seal also aids in maintaining a secure distal placement of the tracheal tube 10 by exerting an expansive force against the tracheal walls, resulting in an interference fit. The proximal end portion 16 of the tracheal tube 10 may then be secured to the patient mouth by using, for example, an endotracheal tube holder and/or tape. The proximal portion 16 of the tracheal tube 10 may include an end connector 24 (e.g., a standard 15 mm, 8.5 mm, or 8 mm end connector, or any suitably sized or configured connector). The end connector 24 includes a lower end suitable for coupling the end connector 24 to the tracheal tube 10 and an upper end suitable for coupling the end connector 24 to a variety of medical airway systems, such as ventilators, manual respirators, suctioning equipment, nebulators, vaporizers, tee connectors, and so forth. It should be noted that some tubes may not include a sealing cuff, or may include more than one cuff, and the lumens in the wall of the tube will be provided and terminated accordingly.

In certain embodiments, the distal end portion 14 and the proximal end portion 16 may be capable of moving axially relative to each other. That is, the proximal end portion 16 may be capable of “sliding” into or out of the distal end portion 14 (or vice versa), so as to allow an extension or retraction in the length of the tracheal tube 10. Such capabilities allow the inflatable cuff 22 to remain securely disposed at a desired distal position inside the trachea. The cuff 22 is capable of remaining at the desired distal position owing to the contact resulting from the expansion of the cuff 22 against the tracheal walls, which will typically be sufficient to allow moving the end portions 14 and 16 relative to each other. Accordingly, the patient may undergo movements of the neck, jaw, and so forth, without dislodging the cuff 22 from its original position. Indeed, such techniques may allow the tracheal tube 10 to maintain a secure and proper seal in the patient airway system, even during activities such as the moving of the patient. The portions of the tube may be similarly moved on purpose, such as when desired to reconnect devices to the proximal end of the tube, to adjust connections and fits of the proximal tube end, and so forth.

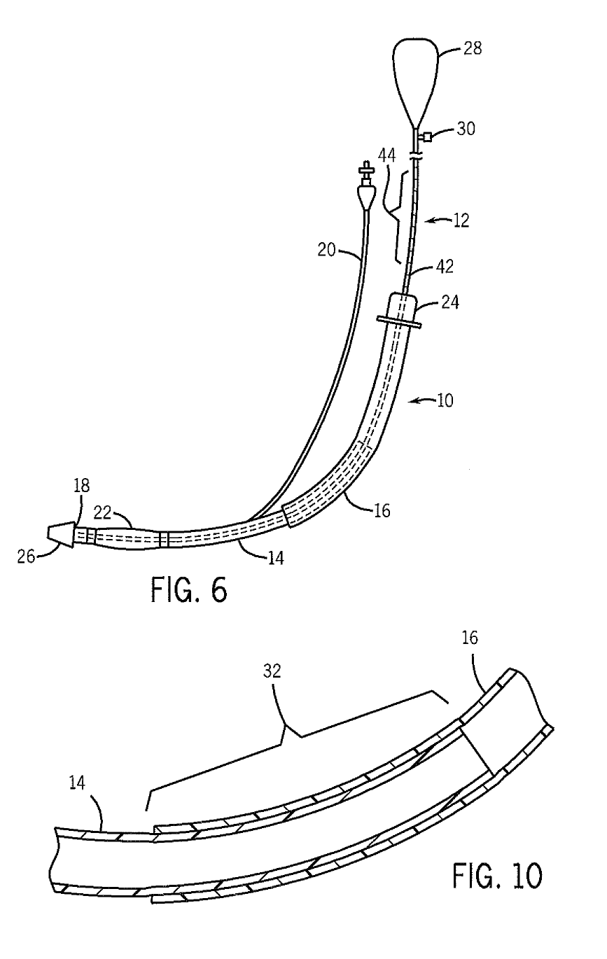

The obturator 12, which may be optional in some cases, may be useful in aiding in the intubation and/or the extubation of the tracheal tube 10. For example, during intubation, the obturator may be inserted into the tracheal tube 10 through the proximal end portion 16 and the distal end portion 14, so as to aid in maintaining rigidity and/or control, such as a torsional or angular positioning and axial rigidity, of the tracheal tube 10. Additionally, the obturator 12 may include techniques such as an inflatable end 26, a pump handle 28, and an air valve 30 suitable for securely attaching the obturator to the tracheal tube 10, as described in more detail below with respect to FIG. 6.

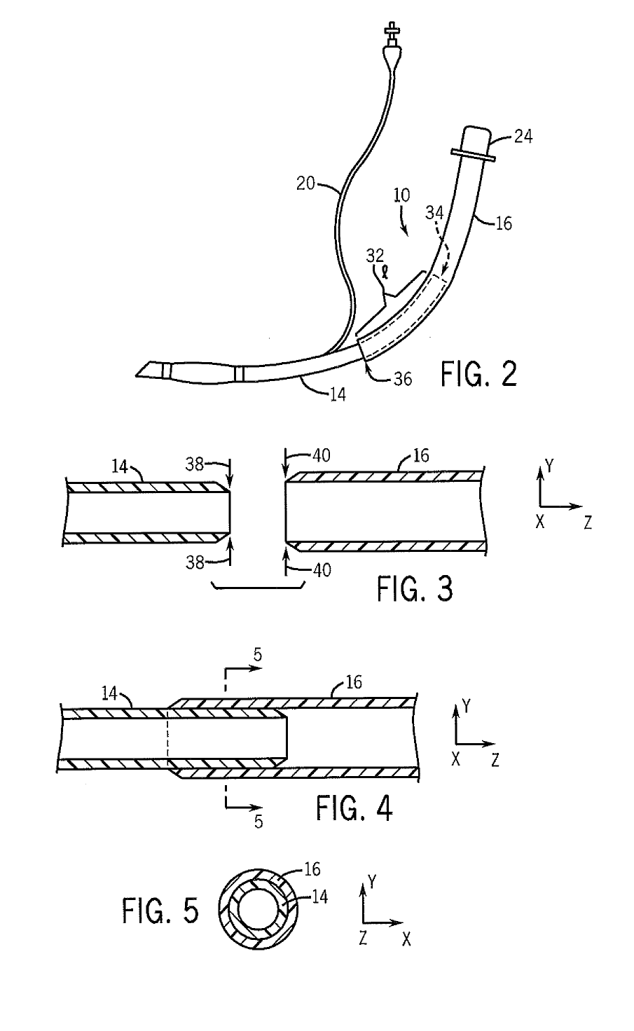

FIG. 2 is a perspective view of an embodiment of the tracheal tube 10 having the movable distal end portion 14 and proximal end portion 16. In the depicted embodiment, the proximal end portion 16 is shown as having a larger outside diameter (OD) than that of the distal end portion 14. Accordingly, a section of the distal end portion 14 is illustrated as being disposed inside of the proximal portion 16. It is to be understood that in other embodiments, the distal end portion 14 may have a larger OD than the proximal end portion 16, and thus, a section of the proximal end portion 16 may be disposed inside of the distal end portion 14. In the depicted embodiment, a section 32 of the tracheal tube 10 having a length l measured from an opening 34 of the distal end portion 14 to an opening 36 of the proximal end portion 16 includes both the end portions 14, 16. As mentioned above, the cuff 22 may be secured to the tracheal walls and the proximal end portion 16 may be secured to the mouth near the end connector 24, thus resulting in the distal end portion 14 and the proximal end portion 16 moving (e.g., “sliding”) axially relative to each other during patient movements. For example, if the patient's neck is extended, then the length/of the section 32 may decrease as the tube openings 34, 36 move inwardly towards each other, increasing the overall length of the tracheal tube 10. Similarly, if the patient's neck is retracted, then the length l of the section 32 may increase as the tube openings 34, 36 move outwardly away from each other, decreasing the overall length of the tracheal tube 10. Indeed, having a variable length section 32 allows the tracheal tube 10 to variable enlarge or reduce its overall length during patient movements while keeping a secure seal around the cuff 22.

In certain embodiments, the OD of the depicted proximal end portion 16 may be approximately 1 mm-15 mm, which may vary depending on whether the patient is a neonatal patient, a pediatric patient or an adult patient. In these embodiments, the inner diameter (ID) of the proximal end portion 16 may be approximately the same diameter as the OD of distal end portion 14, slightly smaller than the OD of the distal end portion 14, or slightly larger than the OD of the distal end portion 14. In certain examples, the ID of the proximal end portion 16 may be within 0.1 mm, 0.25 mm, 0.5 mm, 0.75 mm, 1.0 mm of the OD of the distal end portion 14.

The distal end portion 14 and the proximal end portion 16 may be of any suitable length. For example, the distal end portion 14 may be 50 mm-175 mm in length, and the proximal end portion 16 may also be 50 mm-175 mm in length. Suitable materials for the end portions 14, 16 may include polyvinyl chloride (PVC), polyethylene terephthalate (PET), low-density polyethylene (LDPE), silicone, rubber, polypropylene, acrylonitrile butadiene styrene (ABS), neoprene, polyisoprene, and/or polyurethane. Indeed, the end portions 14 and 16 may be manufactured out of different materials and then assembled together into the tracheal tube 10. In one embodiment, a robotic device or a human operator may assemble the tracheal tube 10 by inserting the distal end portion 14 into the opening 36 of the proximal end portion 16. Accordingly, the tracheal tube 10 arrives at a clinical location with the distal end portion 14 inserted into proximal end portion 16 (or vice versa).

In other embodiments, the tracheal tube 10 does not have the distal end portion 14 inserted during manufacturing. Instead, the clinician is able to first select the end portions 14, 16 suitable for a given type and size of patient and then custom manufacture the tracheal tube 10 in situ by inserting one end portion inside of the other end portion. For example, the clinician may desire to combine a distal end portion 14 having a longer length with a proximal end portion 14 also having a longer length so as to accommodate a larger patient. Accordingly, the clinician may be able to custom fit end portions 14, 16, so as to more comformably fit a variety of patient types (e.g., neonates, children, and adults) and sizes. Further, the end portions 14, 16 may incorporate certain features, such as those described in more detail below with respect to FIG. 3, that allow the clinician to more easily interconnect the end portions 14, 16 as well as allow for an enhanced axial motion (e.g., “sliding”) of the end portions 14, 16.

FIG. 3 is a cross-sectional view depicting embodiments of the end portions 14 and 16 having beveled edges 38, 40, respectively. In the depicted embodiment, the edges 38, 40 are beveled at an angle of approximately 45°. In other embodiments, the edges 38, 40 may not be beveled or may be beveled at more inclined angles (e.g., 35°, 20°, 15°) or less inclined angles (e.g., 50°, 65°, 70°). Additionally, the beveled angle of edge 38 may be different from the beveled angle of edge 40. The beveled edge 38 allows the distal end portion 14 to more easily move in an axial direction along the Z-axis when disposed inside of the proximal end portion 16, because the beveling reduces or eliminates the interference fit at the beveled tip of the distal end portion 14. Additionally, the beveled edge 38 results in an easier insertion of the distal end portion 14 into the proximal end portion 16 by the clinician because the OD of the insertion tip (e.g., distal end portion 14) is smaller than the ID of the receiving tip (e.g., proximal end portion 16) due to the beveled edge 38. Further, the beveled edge 40 allows for the proximal end portion 16 to more easily move with respect to the trachea because the beveled edge 40 may minimize a contact surface and decrease friction if the beveled edge 40 comes into contact with, for example, the lining in the tracheal walls.

FIG. 4 is a cross-sectional view of embodiments of the distal end portion 14 disposed inside of the proximal end portion 16. In the depicted embodiment, the OD of the distal end portion 14 is substantially the same as the ID of the proximal end portion 16. Accordingly, the amount of fluids or gases entering the region between the outside walls of the distal end portion 14 and the inside walls of the proximal end portion 16 may be minimized or eliminated. A slight frictional force between the outer walls of the distal end portion 14 and the inner walls of the proximal end portion 16 may be smaller than the expansive force exerted by the cuff 22 against the tracheal walls. Accordingly, the cuff 22 may stay in place during patient movements while the distal end portion 14 and the proximal end portion 16 move in the Z-axis relative to each other. In other embodiments, the OD of the distal end portion 14 may be slightly smaller, e.g., 0.1 mm, 0.25 mm, 0.5 mm, 0.75 mm, 1.0 mm, smaller than the ID of the proximal end portion 16 so as to allow for the reduction or removal of the frictional force between the end portions 14, 16. In such embodiments, the end portions 14, 16 may move even more freely in the Z-axis with respect to each other. In yet other embodiments, the OD of the distal end portion 14 may be slightly larger, e.g., 0.1 mm, 0.25 mm, 0.5 mm, 0.75 mm, 1.0 mm, larger than the ID of the proximal end portion 16. These embodiments may increase the seal in the region between the outside walls of the distal end portion 14 and the inside walls of the proximal end portion 16, resulting in added protection against fluids or gases leaving the tracheal tube 10. Indeed, the tracheal tube 10 may be manufactured with a variety of properties such as ease of axial movement and leak resistance.

FIG. 5 depicts a cross-sectional Z-axis view of the embodiments of FIG. 4. The figure is illustrative of a co-axial or concentric placement of the distal end portion 14 inside of the proximal end portion 16. In the illustrated example, the co-axial or concentric placement allows for a substantial portion, if not all, of the outer walls of the distal end portion 14 to be circumferentially surrounded by the inner walls of the proximal end portion 16 when inserted into the proximal end portion 16. Accordingly, the amount of fluids or gases entering the region between the outside walls of the distal end portion 14 and the inside walls of the proximal end portion 16 may be minimized or eliminated. It is to be understood that in other embodiments, the proximal end portion 16 may be placed co-axially or concentrically inside of the distal end portion 14.

FIG. 6 depicts embodiments of the extendable tracheal tube 10 and of the obturator 12 that may be used to aid in intubating and/or extubating the tracheal tube 10. The obturator 12 may include a bore 42 having an OD smaller than the ID of the end portions 14, 16. The bore 42 may be of a length greater than the overall length of the tracheal tube 10 when the tracheal tube 10 is fully extended. Additionally, the bore 42 may be semi-rigid (i.e., “bendable”), thus allowing for a clinician to custom fit the bore's curvature manually so as to more comformably fit into the patient's airway. In one intubation example, inflatable end 26 of the obturator 12 may be inserted through the end connector 24 so as to completely traverse the tracheal tube 10, as illustrated. The obturator's inflatable end 26 may then be inflated to an OD larger then the OD of the distal opening 18, causing the inflatable end 26 to cover up the distal opening 18. The inflatable end 26 may be inflated, for example, by closing the air valve 30 and manually squeezing the pump handle 28 so as to pump air through the bore 42 and into the inflatable end 26.

The clinician may then adjust overall length of the tracheal tube 10 by extending or retracting the end portions 14, 16 to a length suitable for intubation into the patient. Indeed, the techniques disclosed herein allow a clinician to custom fit the length of the tracheal tube 10 to more comfortably fit a wide variety of patient types and sizes. Accordingly, the bore 42 may include a plurality of markings 44 along the outside walls of the bore that may be used to determine an overall length of the tracheal tube from the tracheal tube inflatable end 26 to the tracheal tube's end connector 24. Indeed, in certain embodiments, the markings 44 may be found along the entire length of the bore 42. Once the tracheal tube 10 has been adjusted to a suitable length, the clinician may intubate the patient, for example, by using a laryngoscope and by using the obturator 12 as a guide. When the tracheal tube 12 is at a desirable position, the clinician may then open the air valve 30, causing the inflatable end 26 to deflate. The obturator 12 may then be removed from the patient's airway. The tracheal tube 10 may be secured in place by inflating the cuff 22 and by taping the proximal end of the tracheal tube 10 near the end connector 24 to the patient's mouth.

The inflatable end 26 may also be used to reposition the distal end portion 14 while keeping the proximal end portion 16 in place. For example, the clinician may notice during intubation that the distal end of the tracheal tube 10 is either too far up or too far down the patient's airway. Accordingly, the clinician may position the inflatable end 26 inside of the distal end portion 14 and then inflate the inflatable end 26. The inflatable end 26 may then expand against the inner walls of the distal end portion 14, causing a strong interference fit with the distal end portion 14. The clinician may then be able to reposition the distal end portion 14 to a more desirable position by moving the obturator 12 relative to the proximal end portion 16. Such positioning capabilities may allow the clinician to dispose the distal end portion 14 at a precise location in the trachea useful for proper patient airway management.

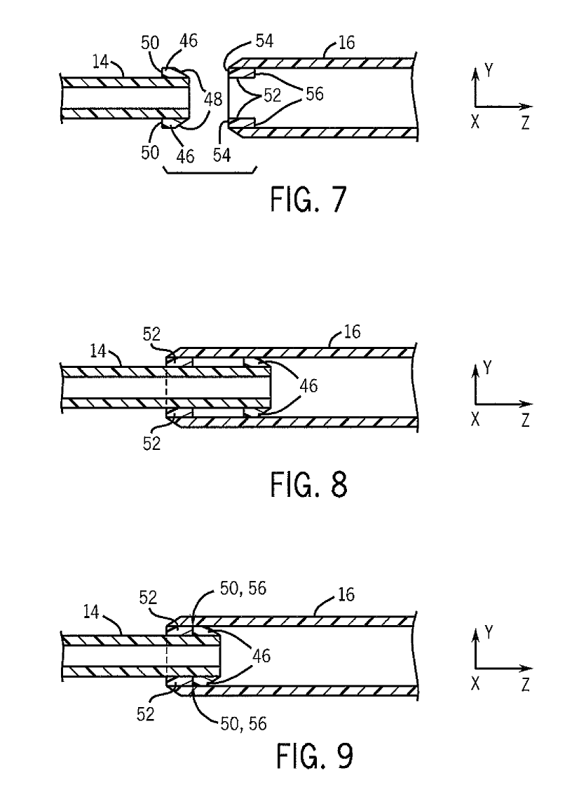

The obturator 12 may also be useful during patient extubation. In one extubation example, the clinician may extubate the patient by first inserting the obturator 12 through the entire length of the tracheal tube 10 until the inflatable end 26 emerges through the distal opening 18. The inflatable end 26 may then be inflated so as to cover up the distal opening 18. The clinician may then extubate the patient by exerting a pulling force on the obturator 12 outwardly from the patient's mouth, causing the inflatable end 26 to lodge against the distal opening 18. The continued exertion of the pulling force in the outwardly direction may then remove the tracheal tube 10 from the patient's airway. Indeed, techniques such as the use of the obturator 12, as well as other techniques discussed in more detail below with respect to FIGS. 7-10, may allow for a rapid and efficient patient extubation.

FIGS. 7-10 are cross-sectional views of embodiments of the distal end portion 14 and the proximal end portion 16 that may allow for a more efficient extubation and that may enhance an attachment between the end portions 14, 16. Turning to FIG. 7, the figure depicts the distal end portion 14 as including a raised edge 46. In the depicted embodiment, the raised edge 46 includes a frontal beveled area 48 and a rear unbeveled area 50. The raised edge 46 may be disposed circumferentially along the outside walls of the distal end portion 14 so as to cover the entire circumference of the tip of the distal end portion 14. The figure also depicts an inner edge 52 disposed circumferentially around the inside walls of the proximal end portion 16. The inner edge 52 includes an unbeveled frontal area 54 and an unbeveled rear area 56. The inner edge 52 may be disposed circumferentially along the inner walls of the proximal end portion 16 so as to cover the entire circumference of the tip of the proximal end portion 16. It is to be understood that in other embodiments, the frontal areas 48, 54 may both be unbeveled or beveled at different angles.

In certain embodiments, such as those depicted in FIG. 8, the edges 46 and 52 are approximately the same height relative to the Y-axis. Having edges 46 and 52 at approximately the same height allows the distal end portion 14 to be placed inside of the proximal end portion 16 (or vice versa), while allowing both end portions 14, 16 to maintain the same ID and OD throughout the length of the tracheal tube 10. That is the edges 46 and 52 are of a height that prevents the creation of compressive or expansive forces that may change the diameters of the end portions 14, 16 in areas near the edges 46, 52. Accordingly, the distal end portion 14 and the proximal end portion 16 may move more smoothly relative to each other along the Z-axis. In other embodiments, the edges 46 and 52 may be of different heights. The different heights may allow for the manufacturing of embodiments capable of increased leak resistance and/or increased ease of axial movements.

As the distal end portion 14 moves in a distal direction and the proximal end portion 16 moves in a proximal direction, the rear area 50 of the edge 46 may come in contact with the rear area 54 of the edge 52, as depicted in FIG. 9. The contact between the edges 46 and 52 may prevent the separation of the end portions 14, 16, from each other. Indeed, the edges 46 and 52 may be capable of securing the attachment between the distal end portion 14 and the proximal end portion 16 so as to allow for the removal of the tracheal tube 10 during extubation. In one extubation example, the clinician may grasp the proximal end portion 16 and exerting a pulling force outwardly from the patient's mouth. The pulling force may result in the movement of the proximal end portion 16 outwardly from the patient airway. Once the edges 46 and 52 come in contact with each other, the pulling force may then also result in the movement of the distal end portion 14 such that both end portions 14, 16 may be extubated out of the patient's airway. Additionally, the obturator 12 may be employed as detailed above with respect to FIG. 6 to further aid in the removal of the tracheal tube 10.

FIG. 10 depicts embodiments of the distal end portion 14 and the proximal end portion 16 where the end portions 14 and/or 16 may be tapered. More specifically, the ID and/or the OD of the end portions 14 and/or 16 may be increasing or decreasing in radial size beginning from a point along the length of the end portion, e.g., the start of the taper. The increase or decrease in radial size then stops at another point further along the length of the end portion, e.g., the end of the taper. Indeed, in certain embodiments, the taper may be included along the entire axial length of the end portions 14 and/or 16. When so tapered, the axial movement of the end portions 14 and 16 away from each other requires ever increase force owing to the tapering creating an ever increasing interference fit between the two end portions 14, 16. Accordingly, as the section 32 becomes smaller (i.e., the length/of section 32 is decreasing), the interference fit becomes stronger. FIG. 10 shows an embodiment where the increase of the interference fit is manifested by a slight inward compression of at the tip of the distal end portion 14 and a slight outward expansion at the tip of the proximal end portion 16. Indeed, such interference fit may become quite substantial, thus aiding the clinician in extubating the patient. The patient may be extubated by manually grasping the proximal end portion 16 and exerting a pulling force outwardly from the patient's mouth. As the proximal end portion 16 moves axially away from the distal end portion 14, the interference fit grows stronger. Eventually, the interference fit becomes strong enough so as to securely attach the distal end portion 14 to the proximal end portion 16, thus allowing the removal of both end portions 14, 16 from the patient's airway. Indeed, the techniques disclosed herein allow for a quick and efficient extubation of the tracheal tube 10 while maintaining a secure attachment between the distal end portion 14 and the proximal end portion 16. Further, the techniques disclosed herein are capable of the secure and proper placement of the tracheal tube 10 while allowing for a variety of patient movements. Such capabilities may result in a more efficient and comfortable airway management and ventilation.

What is claimed is:

1. An extendable tracheal tube system comprising:
a tracheal tube comprising a distal end portion; and
a proximal end portion configured to be coupled to the distal end portion,
wherein the proximal end portion and the distal end portion are configured to move axially relative to each other when positioned in an airway of a patient.

2. The system of claim 1, wherein the distal portion of the tracheal tube system comprises a cuff.

3. The system of claim 1, wherein a first section of the distal end portion is disposed co-axially inside of a second section of the proximal end portion.

4. The system of claim 1, wherein a first section of the proximal end portion is disposed co-axially inside of a second section of the distal end portion.

5. The system of claim 3, wherein an outside diameter of the distal end portion is approximately equal to an inner diameter of the proximal end portion.

6. The system of claim 4, wherein an outer diameter of the proximal end portion is approximately equal to an inner diameter of the distal end portion.

7. The system of claim 1, comprising an obturator configured to traverse the proximal portion, and the distal portion.

8. The system of claim 7, wherein the obturator comprises an inflatable end.

9. The system of claim 1, wherein the distal end portion comprises a raised edge and the proximal end portion comprises an inner edge, wherein the raised edge and the inner edge come in contact with each other when the distal end portion is moved in a distal direction or the proximal end portion is moved in a proximal direction.

10. The system of claim 1, wherein either the distal end portion, the proximal end portion, or both end portions comprise a taper along a length of the end portion, wherein the taper increases an interference fit between the distal end portion and the proximal end portion as the distal end portion is moved in a distal direction or the proximal end portion is moved in a proximal direction.

11. The system of claim 1, wherein the tracheal tube is an endotracheal tube or a tracheostomy tube.

12. An extendable tracheal tube system comprising:
a tracheal tube comprising a distal end portion and a proximal end portion receiving one another and configured to move axially relative to one another;
an end connector configured to be coupled to the proximal end portion at a lower end and to a ventilator conduit at an upper end; and
a cuff coupled to the distal end portion and configured to be expanded to contact a patient airway.

13. The system of claim 12, wherein a first force required to decouple the cuff from the patient airway is greater than a second force required to move the proximal end portion and the distal end portion relative to each other.

14. The system of claim 12, wherein the proximal end portion comprises a first beveled edge and the distal end portion comprises a second beveled edge.

15. The system of claim 14, wherein the first beveled edge is at a different bevel angle from the second beveled age.

16. The system of claim 12, wherein the distal end portion comprises a raised edge and the proximal end portion comprises an inner edge, wherein the raised edge and the inner edge come in contact with each other when the distal end portion is moved in a distal direction and the proximal end portion is moved in a proximal direction.

17. The system of claim 12, comprising an obturator configured to traverse the end connector, the proximal portion, and the distal portion.

18. A method of manufacturing an extendable tracheal tube system comprising:
manufacturing a distal end portion having a first length; and
manufacturing a proximal end portion having a second length, wherein the proximal end portion and the distal end portion are configured to be coupled in fluid communication with each other and to move axially relative to each other.

19. The method of claim 18 wherein the distal end portion and the proximal end portion are manufactured out of polyvinyl chloride (PVC), polyethylene terephthalate (PET), low-density polyethylene (LDPE), silicone, rubber, polypropylene, acrylonitrile butadiene styrene (ABS), neoprene, polyisoprene, polyurethane, or a combination thereof.

20. The method of claim 18, comprising manufacturing an obturator comprising an inflatable end, wherein the obturator is capable of traversing the proximal portion and the distal portion of the extendable tracheal tube system.
